(19) United States
(12) Patent Application Publication
(10) Pub. No.: US 2009/0016930 A1
(43) Pub. Date: Jan. 15, 2009

(54) SANITIZATION USING HIGH ELECTRIC FIELDS

(75) Inventor: Eugene I. Gordon, Mountainside, NJ (US)

Correspondence Address: DARBY & DARBY P.C., P.O. BOX 770, Church Street Station, New York, NY, 10008-0770, US

(73) Assignee: Germgard Lighting, LLC, Dover, NJ (US)

(21) Appl. No.: 12/172,870

(22) Filed: Jul. 14, 2008

Related U.S. Application Data
(60) Provisional application No. 60/949,766, filed on Jul. 13, 2007.

(51) Int. Cl. A61L 2/00

(52) U.S. Cl. 422/22; 422/292

(57) ABSTRACT

A sanitizer utilizing electrical fields for hands and gloved hands. The sanitizer includes a power supply, a first and second electrode, and a dielectric between the first and second electrode. For sanitizing the outer surface of a glove, the first electrode is disposed inside the glove and the second electrode is disposed near the outer surface of the glove. A signal generator produces a high-voltage, periodic signal across the first and second electrode for a predetermined period of time to generate an electrical field configured to inactivate pathogens between the two electrodes. The second electrode can include a metallic layer or a conducting liquid in which a gloved hand is inserted. Alternatively, an electric field can be generated between the stratum corneum of a user's skin and an outer conducting layer of a glove so as to deactivate pathogens on the surface of the hand.

This application claims priority under 35 U.S.C. § 119(e) to U.S. Provisional Patent Application Ser. No. 60/949,766, entitled “Sanitation System Using Electrical Fields and Metallic Coatings” and filed Jul. 13, 2007, which is hereby incorporated by reference as though set forth in its entirety herein.

### FIELD OF THE INVENTION

The present invention relates to sanitization using electric fields, and more specifically to the sanitization of a surface of a gloved hand by generating an electric field across the glove so as to destroy pathogens present on the surface.

### BACKGROUND OF THE INVENTION

It is increasingly accepted that the dominant source of infection in a hospital or surgical environment is associated with the pathogens that reside on virtually all surfaces including the clothing of doctors and healthcare workers, medical instruments, and the patients themselves. Patients can transfer pathogens to the hands and clothing of doctors or healthcare workers, which can in turn be transferred to other surfaces by contact.

Surfaces are a vast storehouse of infectious pathogens, which can survive on those surfaces for months. When a patient comes in contact with a doctor, some of these pathogens on the patient are transferred to the hands and clothes of the doctors and healthcare workers. As doctors and healthcare workers move through the hospital and/or office, these pathogens can be transferred to other patients or the patient surround (i.e., the general surroundings of the patient, such as door handles, walls, medical instruments, exam tables, magazines, pens, etc.) by further contact. The pathogens move from one area to another (e.g., room to room, surface to surface, bed to bed, and patient to patient) on hands and clothing, and in the air.

Prevention of the spread of pathogens can be increased if the healthcare workers and doctors frequently sanitize their hands, ideally just before a patient or the patient surround is contacted. As a practical consequence of the multiple sanitations requirement, if hand sanitation is practiced it must be performed locally, quickly, conveniently, effectively, and without damaging or irritating the skin of the hands. Preferably, the cost of sanitation should not be high.

Currently mechanisms for addressing this problem are limited. One method of inactivating pathogens on the surface of the gloved hand using UVC illumination of the glove surface to achieve gloved hand sanitation during patient care or surgery is described in U.S. patent application Ser. No. 12/109,141, filed Apr. 24, 2008 and assigned to the present assignee, which is hereby incorporated by reference as though set forth in its entirety herein. The inactivating procedure is carried out in seconds in a small, closed box containing UVC illumination. The box is preferably positioned near the patient. The gloves are opaque to the UVC and protect the skin of the hand from exposure; hence the skin is not damaged by the UVC. Since the hands of the healthcare worker or doctor are frequently gloved to protect themselves from the patient, gloving is not an onerous requirement.

Gloved hand sanitation is therefore possible but not yet available, nor has it been adopted by the industry or healthcare community. Improvements in efficacy, convenience, and speed as well as a reduction in cost are needed to ensure adoption by the industry and demand by the healthcare community and consumers.

An alternative method of inactivation of pathogens is by exposing pathogens to an electric field. This technique is known for liquid food, such as milk and soup, as an alternative to thermal inactivation (i.e., pasteurization). The U.S. Food and Drug Administration, Center for Food Safety and Applied Nutrition published a summary report on this technique on Jun. 2, 2000 entitled Kinetics of Microbial Inactivation for Alternative Food Processing Technologies; Pulsed Electric Fields.

One mechanism for inactivation described in the FDA report is electroporation (i.e., electrocompression), which involves a liquid surround that is preferably somewhat conducting. To date all such pathogen inactivation studies have been conducted in relatively large volumes requiring that the necessary high electric fields be produced using repetitive charging and pulsing, capacitive discharge or ladder networks, with the pathogen immersed in a slightly conducting medium. High electric fields produced by opposing plates with the pathogens in a slightly conduction medium is compression of the membrane enclosing the pathogen by an induced, high electric field within the membrane. Stated simplistically, the externally applied electric field separates mobile charges within the body of the pathogen, moving them to opposite inner walls of the membrane as described above. This charge redistribution shields the electric field within the pathogen, eliminating the electric field internal to the pathogen.

If the surrounding medium is electrically conducting, the electric field between plates and the charge on the inner membrane wall attracts free charges of the opposite sign in the medium surrounding the pathogen to the outer surfaces of the membrane wall. Depending on the dielectric constants of the various elements, and the conductivity, the resulting electric field within the membrane can be very high. The two opposing layers of opposite charge on the membrane wall, a dipole layer, attract one another and the attractive force puts the membrane wall under compression. If the applied electric field is large enough and the medium conductivity high enough, the resulting high field causes the membrane to break up and to leak internal fluid to the outside, leading to death of the pathogen. A discussion of the process can be found in an article by Professor Shesha Jayaram, entitled “Sterilization of Liquid Foods by Pulsed Electric Fields,” and published in the IEEE Electrical Insulation Magazine, Vol. 16. No. 6, November/December 2000.

If the surrounding medium is not conducting then no charge is attracted to the outer wall of the membrane. The electric field terminates on the charges attracted to the inner surface of the membrane as shown below. This greatly reduces the field across the membrane and greatly reduces the force compressing the membrane. Hence the same electro-phoretic mechanism exists but is greatly reduced in its ability to compress the membrane.

The other mechanism utilizing electric breakdown and ionic punch-through is a dry mechanism. In this mechanism, the cell membrane can be considered to be a capacitor filled with a leaky dielectric. The natural potential difference across the cell is about 10 mV. In liquids such as milk, when an external field E>10,000 V/cm is applied, a pathogen cell having a dimension of about 1 micron initially experiences a voltage across it of about 1 volt. This induces free charge motion within the cell, whereby the charge redistributes itself about the inner surface of the cell membrane until the applied electric field internal to the cell is cancelled out. The cell acts as a typical isolated conductor in an applied electric field. That is, it cancels out the applied field internally because it cannot support an applied, internal electric field. There is no path for conduction current, only displacement current when the electric field varies. According to the existing research, a pulsed electric field of at least 10,000 V/cm must be used to destroy pathogens. A field strength below 10,000 V/cm does not destroy pathogens.

What is needed in the art is an effective, simple, and cost-efficient way to utilize electric fields to sanitize the surface of a hand or a gloved hand.

### SUMMARY OF THE INVENTION

In accordance with the present invention, a gloved hand sanitizer utilizing electrical fields is provided. The gloved hand sanitizer includes a power supply, an inner glove, a first electrode, a second electrode, and a slightly conducting dielectric disposed between the first and second electrode. The first electrode is disposed inside the glove and the second electrode is disposed near the outer surface of the glove so as to decrease the space between the first and second electrode. A portion of the glove acts as a leaky dielectric between the first and second electrode. A signal generator is connected to the first and second electrode so as to generate a high-voltage, periodic signal across the first electrode and the second electrode for a predetermined period of time. The high-voltage periodic signal generates an electrical field configured to inactivate pathogens disposed between the first electrode and the second electrode.

In a further aspect of the present invention, the second electrode is disposed within a rigid structure so as to define a volume in which the gloved hand can be inserted and a volume between the second electrode and the rigid structure. A pressure device is coupled to the rigid structure so as to allow the removal or addition of air or other gas into the volume between the second electrode and the rigid structure. When gas is added to the volume, the second electrode deforms around the gloved hand so as to bring the second electrode in close proximity to the first electrode.

In yet a further aspect of the present invention, the second electrode can comprise a conducting fluid. The gloved hand can be inserted in to the fluid so as to bring the second electrode in close proximity to the first electrode. One possible conduction fluid is a salt water solution. Preferably a non-wetting conduction fluid, such as Mercury, can be used. However, Mercury is not currently practical in view of certain environmental and safety concerns.

A further aspect of the present invention provides for sanitization of the surface of a bare hand. The hand can be inserted in a glove having an electrode on the outside of the glove. The wet skin of the hand can be used as a surface conductor. Thus, a signal generated through the glove's conductor produces an electric field configured to deactivate pathogens on the surface of the hand.

These and other aspects, features and advantages will be apparent from the following description of certain embodiments and the accompanying drawing figures.

### BRIEF DESCRIPTION OF THE FIGURES

FIG. 1 illustrates the charge distribution on an isolated, uncharged conductor in a uniform electric field;

FIG. 2 illustrates a simplified embodiment of the present invention;

FIG. 3a illustrates an embodiment of the present invention including an inner and outer glove;

FIG. 3b illustrates a cross section of the inner and outer glove illustrated in FIG. 3a;

FIG. 4 illustrates a further device for sanitizing a gloved hand in accordance with an embodiment of the present invention;

FIG. 5 illustrates an embodiment of the present invention that uses a non-wetting, conducting liquid as an outer second electrode; and

FIG. 6 illustrates a cross section of an embodiment of the present invention that utilizes a user's skin as a surface conductor.

### DETAILED DESCRIPTION OF CERTAIN PREFERRED EMBODIMENTS

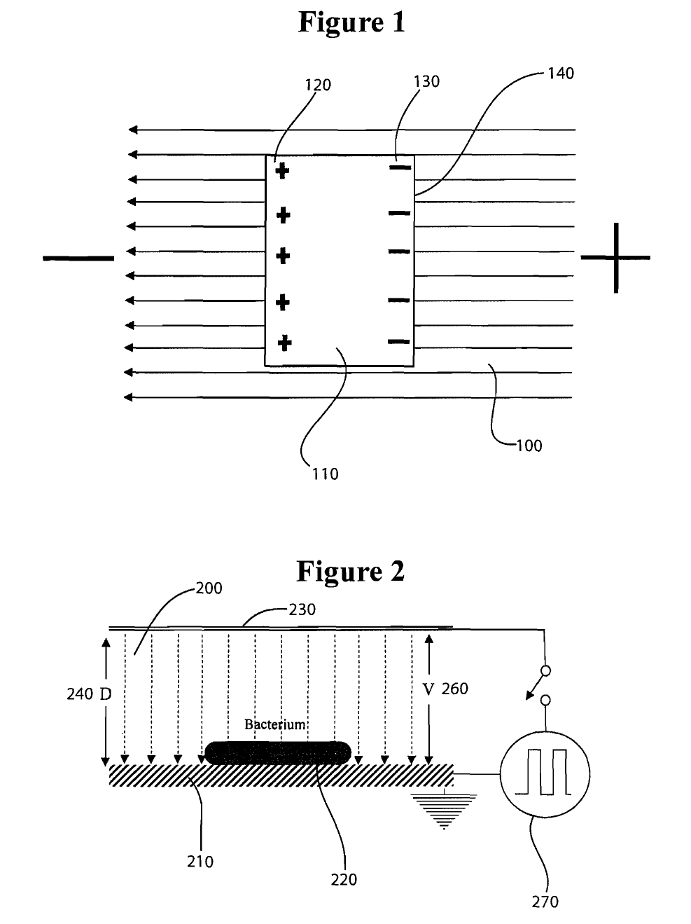

By way of overview, FIG. 1 illustrates an exemplary charge distribution on an isolated, uncharged conductor 110 in a uniform electric field 100. Not being charged, the net electric field force on the conductor is zero. However, the free charges (i.e., positive charges 120 and negative charges 130) within the conductor are attracted to the surface 140 of the conductor by the external field 100, which then exerts forces tending to pull the conductor 110 apart.

FIG. 2 illustrates a typical pathogen 220 on a grounded, flat metal plate 210 with an opposing flat metal plate 230 spaced at a distance of D (cm) 240. Air fills the space between the plates 250. A voltage V 260 (Volts) is applied to the opposing plate 230 producing a uniform electric field 200 across the gap space D 240 having a magnitude E=V/d (Volts/cm). The field can be applied for a time, τ (sec.). If the electric field 200 strength exceeds a critical value for that type of pathogen 220, and if τ is long enough, the pathogen will be destroyed. The critical value of electric field 200 depends on the particular type and size of pathogen 220 and the temporal behavior of the applied electric field 200. Preferably, a conducting medium (e.g., a conducting fluid) is in close proximity to the pathogen, thereby ensuring its destruction.

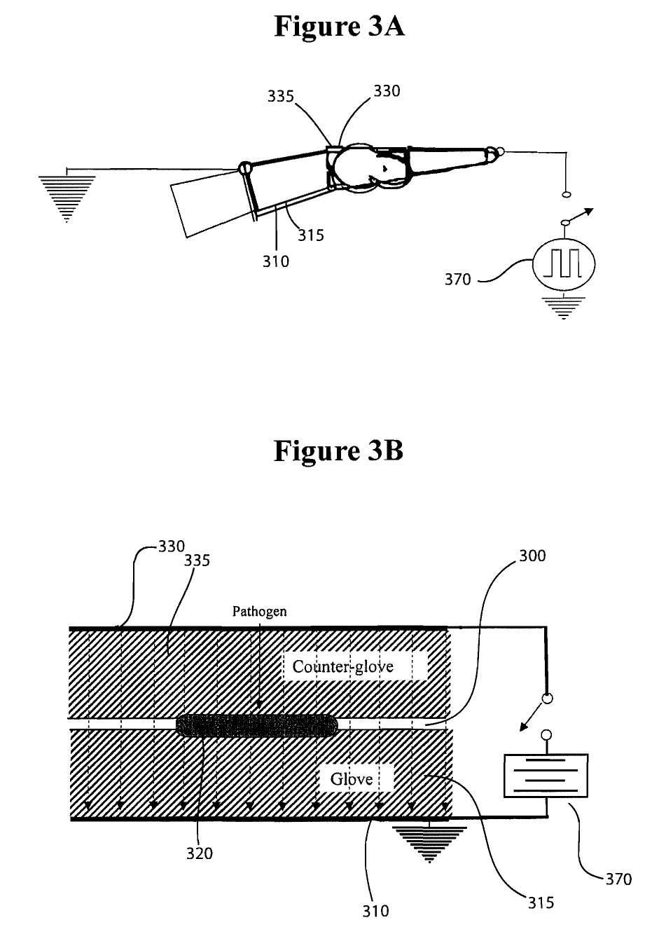

By applying the necessary electric field across the surface of a glove, the pathogens on the surface can be destroyed, thereby sanitizing the glove surface. FIG. 3a is a glove sanitization system in accordance with the present invention, and FIG. 3b is a cross section of the exam glove 315 and outer glove 335 in accordance with the present invention, wherein like references numbers refer to like elements.

A user wears a glove 315 having a typical thickness of approximately 125 microns to about 25 microns. The inner surface of the glove is coated with, or consists of, a thin layer of metal layer 310. Alternately, a highly conducting gel coating could be placed on the hand under a standard glove so as to act as the ground plane, thus eliminating the need for a special exam glove, similar to the glove discussed below.

The exam glove can then be fitted into a similar outer glove 335 having a conducting layer 330 on the outer surface of the outer glove 335. Pathogens 320 residing on the surface of the glove 315 are effectively within a sandwich of two conducting layers 310 and 330, separated by two slightly conducting dielectric layers 315 and 335. Each glove's dielectric layers can be approximately 125 microns (i.e., five mils) thick, so the total spacing between the conducting layers is about 250 microns. Alternatively thicknesses are possible depending on the strength of the material used. Particularly, it should be noted that the outer glove 335 does not need to be as thick as the inner glove, since it will not be used during an examination. Therefore, the thickness of the outer glove 335 can be reduced relative to the exam glove 310.

It is known that if an applied electric field strength in the region of the pathogen exceeds a critical value for that type of pathogen, and if τ is long enough, the integrity of the membrane of the pathogen in the electric field will be destroyed allowing internal fluid to leave. The value of critical electric field depends on the particular type, shape, and size of pathogen. However, field strength in the range 30-50 kV/cm should be sufficient to destroy most pathogens. If the applied field sufficiently exceeds the critical field, τ would be only a few milliseconds at most. A target field value is 40,000 Volts/cm.

Spacing between metal layers on the order of 250 microns (i.e., 10 mils), would require an alternating voltage of order 500 volts between the glove electrodes. The current is preferably low, thereby dissipating negligible power. As small solid state circuit operated from a battery would be sufficient to generate the required voltage.

A voltage V (Volts) applied to the opposing plate 330 by signal source 370, produces an electric field 300 across and within the space between the dielectric layers 315 and 335. The field 300 is applied for a time equal to τ (sec.). Because the pathogen dimensions are on the order of 1 micron, a voltage of approximately 4 volts across the pathogen—enough to destroy it—would be approximately 40,000 V/cm. Given the distance between the two conducting layers 310 and 330, the signal source would only need apply a few hundred volts (e.g., 300-500 Volts). The field 300 applied for time τ would effectively sanitize the surface of the glove. Although the term sanitation is used the remaining fraction of pathogens can be in the range of −6 log₁₀, i.e., sterilization. Additionally shielding and clumping of pathogens would not reduce the destruction rate, and a very brief exposure would eliminate virtually all pathogens on the surface of the glove.

An electric field applied across a chamber with a substantial volume requires tens of thousands of volts across large area plates to produce the necessary field strength. However, by reducing the distance between the two conducting layers 310 and 330, the voltage required is greatly reduced.

Pulsing is a convenient way to obtain the necessary high voltages and charging currents. Various pulsed waveforms can be used, but one particularly effective waveform was a sequence of approximately square wave voltage pulses obtained using a standard square wave pulse forming network with a series inductor and parallel capacitor in a repetitive ladder. The equivalent, or cumulative, exposure time for a sequence of square wave voltage pulses is approximately the sum of the pulse lengths. With respect to the discussion contained herein, we do not distinguish the application of a series of square wave pulses from the application of DC voltage for a fixed exposure time approximately equal to the sum of the pulse widths. It would be known by one of ordinary skill in the art that a DC voltage, a series of square pulse, or an equivalent can be used to achieve the necessary field strength.

The external electric field 300 terminates on and exerts forces on the charged elements at the inner and outer surface of the pathogen membrane 320. These forces, if large enough, begin to pull the pathogen membrane apart. Irreversible breakdown occurs when the maximum tolerable compressive forces on the membrane are exceeded for a long enough time, which is typically 10 microseconds to 1 millisecond. The forces cause the formation of trans-membrane pores which allow fluid discharge and decomposition of the membrane. Above the critical field strength and over time, larger areas of the membrane develop pores with mechanical destruction of the cell membrane; consequently cell death occurs.

In a group of pathogens the fraction of remaining active pathogens decreases with time and a remaining fraction as small as 10⁻⁷ can be achieved. The critical field value also depends on the temporal evolution of the pulse. For pulsed voltages it depends on the pulse length, short pulses of a few microseconds have critical fields greater than or equal to 30,000 V/cm, while much longer pulses have critical fields less than 15,000 V/cm.

As discussed above, the user wears an exam glove 315 having a ground layer 310. A woven copper ground glove is preferable and provides a good fit for the individual user and fitting well up on the forearm. The glove 315 is then grounded, and the regular exam glove 315 either simply pulls over the ground layer 310 or the ground layer is integrally formed with glove 315. The conductive layer acts as a Faraday cage to electrically protect the hand within the glove and further provides the ground plane for an applied external electric field passing through the exam or surgical glove. Accordingly a large amplitude voltage can be applied between a conducting layer (electrode) at the inner surface of the exam glove and a second conducting surface (electrode) placed over the exam glove outer surface in intimate contact with the surface preferably wetted by a slightly conducting fluid.

Optionally, the inner surface of the exam glove 315 can include a flexible, conforming, conducting coating as the ground layer 315. Alternatively, as discussed above, a highly conducting gel can be placed on the hand to act as the ground plane, thus eliminating the need for a special exam glove.

Using copper to line the exam glove can provide additional sanitizing features. Pathogens on a copper surface are quickly inactivated to a considerable extent. Coating or lining the inner surface of the glove with a thin copper layer allows skin sanitation and protection against pathogens penetrating the glove. The danger of leakage of pathogens from the outer surface through a porous glove can be considerably reduced by virtue of the sterilizing action.

The time required to achieve 10⁻⁷ inactivation by copper is about 90 minutes. While the time required to achieve significant pathogen inactivation is far too long for use of copper or other media to sanitize bare hands, hands inside copper lined exam gloves for extended periods, and reasonably protected against new pathogens coming onto the skin surface it, the copper lining can provide a considerable level of sanitation and protection. It also makes sanitation of the bare hand between patient visits unnecessary. Thus, the gloves can be used over a somewhat extended period requiring only sanitation of the external surface. Additionally, this would eliminate skin irritation, save time between patient visits and provide protection against pathogens getting through the glove. However, extended wear has some negative features such as sweating and maceration of the skin so this aspect depends on the particular glove,

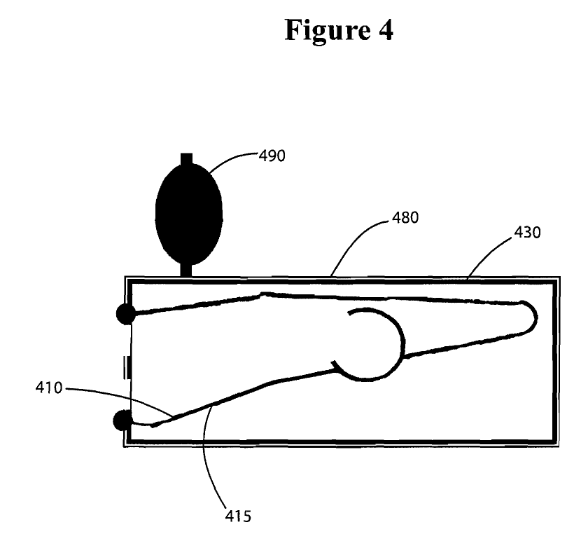

FIG. 4 illustrates one way by which the exam glove 415 can be quickly placed inside the outer electrode glove 430. The outer electrode glove 430 is highly elastic and made of conducting material. It is preferably substantially air tight and the forearm end is fitted to the opening of a box 480, which can support a partial vacuum or a slight overpressure. The partial vacuum or the slight overpressure can be created externally by a small pump and a ballast volume 490. The pump 490 can create partial vacuum or slight overpressure as required. Under partial vacuum the outer electrode glove 430 balloons out under the force of the atmospheric pressure within the glove. This produces a substantially oversized, outer electrode glove 435 preferably with stretched apart finger receptacles. The exam glove 415 having a conducting layer 410 is easily inserted into the oversized outer electrode glove 430. The air pressure in the box can then be changed to slightly above atmospheric pressure using the pump 490, and the elastic outer glove 430 would shrink tightly onto the exam glove 415, thereby making close contact.

The spacing between the inner conducting layer 410 of the exam glove 415 and the outer conducting electrode glove 430 (i.e., the electrode spacing) is determined approximately by the thickness of the exam glove 415. The exam glove 415 is a dielectric with high resistance and conducts only displacement current. The surface is preferably wetted with a conducting fluid. A voltage such as described above is then applied to the two electrodes defined by the conducting outer glove 430 and the conducting ground layer 410 at the inner surface of the exam glove 415. The resulting electric field (not shown) is directed roughly normal to the two exam glove surfaces. Any pathogen on the insulating outer surface of the wetted exam glove 415 would be exposed to the applied high electric field despite any irregularities in the surface of the exam glove. In a fraction of a second all types of pathogens on the surface of the exam glove 415 would be destroyed to a remaining fraction level of 10⁻⁶ or better. Applying the partial vacuum again within the box 480 would allow the outer electrode glove 430 to expand and the exam glove 415 to be removed. The electrode glove 430 optionally remains in the expanded state until the next use.

One of ordinary skill in the art should know that the pump 490 can include or a single stroke piston or other device for creating a partial vacuum or slight overpressure as required.

Using this approach the sanitization device can be battery operated and small enough to be attached to the uniform of the healthcare worker or doctor if desired. Sanitization could be achieved very fast (e.g., within a sub second interval for killing of most pathogens) and can be used an unlimited number of times without damage or irritation of the skin. Hence multiple sanitations during a patient visit are possible, effectively maintaining the bare hand or glove in a sterile state and protecting the patient and the healthcare worker.

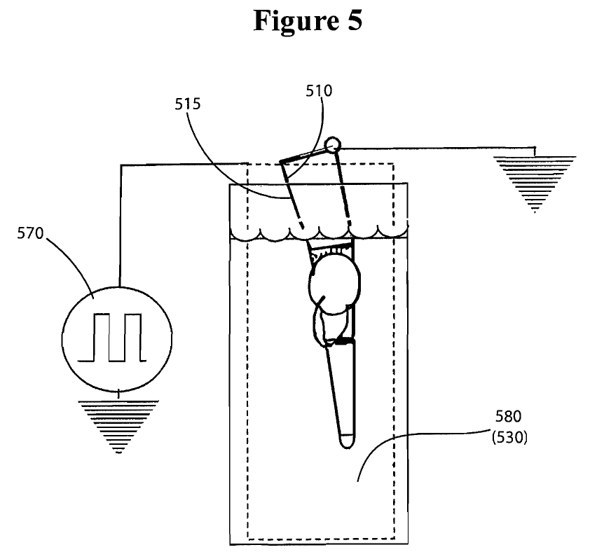

A further approach, shown in FIG. 5 requires the use of a conducting liquid 580. The conducting, liquid 580 would conform exactly to the outer surface of the exam glove and serve as the outer second electrode 530. Preferably the liquid 580 is non-wetting with respect to the material of the exam glove 515 and is highly conducting. A non-wetting liquid would not stick to the exam glove 515. However, wetting liquids (e.g., salt water) can be utilized as well.

Mercury would be an ideal non-wetting, liquid conductor, but there are strict limitations on the use of mercury. Although mercury is ideal in all other respects its vapors and certain of its compounds are poisonous. Thus, the environmental and safety precautions necessary for its use substantially reduce its practicality. However, alternate fluids or mixtures of fluids and small conductors that are both conducting and non wetting might be found in the future. One wetting fluid would be salt water. A quick snap of the gloved hand might be adequate to leave the surface relatively free of fluid.

In the context of sterilization of a glove 515 with an inner conductive coating 510, the example is given for a grounded inner conducting layer 510 using a simple grounded conducting clip 590 connected to the inner glove surface. However other variations are possible. For example, an inner glove 510 made of finely woven metal, coated with a nitrile layer to provide a pathogen barrier. The conductive layer 510 would serve as a Faraday cage to electrically protect the hand within the exam glove and provide the ground plane for the external electric field passing through the exam glove.

In a further aspect of the present invention it is possible to use the skin of the bare hand as a conducting element. Bare skin has electrical resistance that depends on its condition, such as cleanliness and dryness. The conductivity can be increased by immersion in salt water. The resistivity is a function of depth below the surface of the skin.

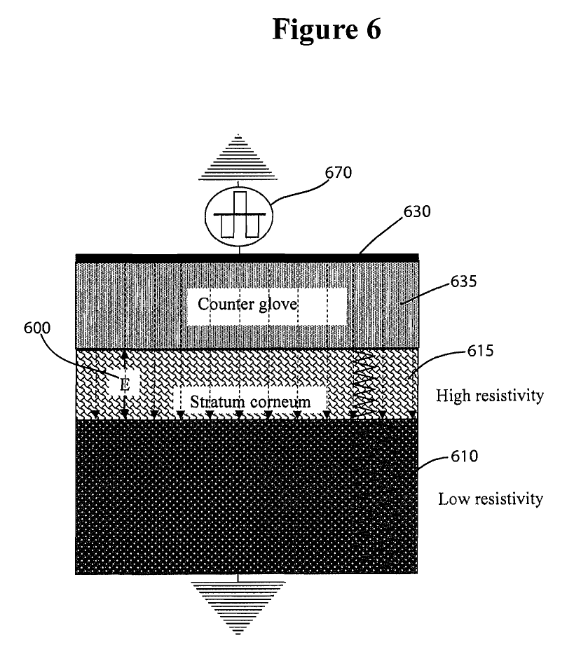

FIG. 6 is an illustrative cross section of a portion of a hand inserted into an outer glove 635 without and exam glove disposed between them. The stratum corneum layer 615 of the human skin, about 20 to 40 microns thick, is fairly resistive. Accordingly, with a non conducting, dielectric material counter glove 635 backed by a good conductor 630, it should be possible to produce a pulsed electric field across the counter glove dielectric 635 and stratum corneum 615 terminating within the epidermis 610. Thus, the outer glove and the skin act as the two electrodes between which the field is generated.

A square wave voltage applied across the conducting layer 630 of the counter glove 635 and the skin 610 produces a voltage split across the counter glove dielectric 635 and the stratum corneum 615. The glove dielectric 635 and the stratum corneum 615 behave similarly to two capacitors connected in series, and an electric field appears across each of the glove dielectric 635 and the stratum corneum 615. Accordingly, the electric field generated within the stratum corneum 615 peaks, and then decays as a function of the resistance of the stratum corneum. If the electric field generated in the stratum corneum is large enough, it will destroy pathogens within the stratum corneum.

A field of 40,000 volts/cm across a 20 micron thick layer of stratum corneum 615 would require a voltage drop of 80 volts. While that field strength and voltage, if maintained across the stratum corneum, could conceivably damage the skin depending on its resistivity, once the current begins flowing through the slightly conducting stratum corneum 615, thereby discharging the capacitance of the layer, the electric field across the layer decays. Transient pathogens at or near the outer surface of the layer would be destroyed. As such pathogens would experience a high continuous electric field. The decaying electric field within the stratum corneum 615 would also destroy pathogens beneath the surface.

While the present invention has been particularly shown and described with reference to preferred embodiments thereof, it will be understood by those skilled in the art that various changes in form and details may be made therein without departing from the spirit and scope of the present invention.

We claim:

1. A hand sanitizer, comprising:
a power supply;
an inner glove having an inner surface and an outer surface;
a first electrode adjacent to the inner surface of the inner glove;
a second electrode, selectively disposable next to the outer surface of the inner glove;
a dielectric, the dielectric including at least a portion of the inner glove and being disposed between the first electrode and the second electrode; and
a signal generator, the signal generator configured to generate a high-voltage, periodic signal across the first electrode and the second electrode for a predetermined period of time, such that the high-voltage periodic signal generates an electrical field configured to inactivate pathogens disposed between the first electrode and the second electrode.

2. The hand sanitizer of claim 1, further comprising a layer of conducting fluid disposed on the outer surface of the inner glove.

3. The hand sanitizer of claim 1, wherein the first electrode is integrated into the inner glove, and the second electrode includes a conducting layer and an outer glove, the outer glove having an inner surface and an outer surface wherein the inner surface of the outer glove is selectively disposable next to the outer surface of the inner glove, and the conducting layer of the second electrode is disposed next to the outer surface of the outer glove.

4. The hand sanitizer of claim 3, wherein the dielectric comprises a first layer of at least one of the inner glove and the outer glove, the first layer being at least slightly conducting.

5. The hand sanitizer of claim 1, wherein the second electrode comprises a conducting fluid.

6. The hand sanitizer of claim 5, wherein the conducting fluid is non-wetting.

7. The hand sanitizer of claim 1, wherein the field generated has a strength of approximately 30 kV/cm to 50 kV/cm.

8. The hand sanitizer of claim 1, wherein a frequency of the high-voltage periodic signal is approximately 10 kHz to 1,000 kHz.

9. The hand sanitizer of claim 1, wherein an amplitude of the high-voltage periodic signal is approximately 80 V to 500 V.

10. The hand sanitizer of claim 1, wherein the period of time is less than approximately 1,000 ms.

11. The hand sanitizer of claim 1, wherein a distance between the first electrode and the second electrode when the second electrode is disposable next to the outer surface of the inner glove is less than about 250 microns.

12. The hand sanitizer of claim 1, further comprising:
a rigid enclosure, the second electrode comprising a deformable material and disposed within the rigid enclosure so as to define a first volume between the rigid enclosure and the second electrode; and
a pressure device coupled to the rigid enclosure and configured to adjust a pressure level within the first volume so as to deform the second electrode,
wherein, when the inner glove is inserted in the box and the pressure level of the volume is increased, the second electrode is disposable substantially against the outer surface of the gloved hand.

13. A method of sanitizing an outer surface of a surface of a glove worn by a person, comprising the steps of:
disposing the glove to be sanitized between a first electrode and a second electrode, the first electrode disposed substantially next to an inner surface of the glove and the second electrode disposed substantially next to the outer surface of the glove;
applying a high-voltage periodic signal across the first electrode and the second electrode for a period of time;
generating a field using the high-voltage periodic signal, the field being configured to inactivate pathogens disposed between the first electrode and the second electrode.

14. The method of claim 13, wherein the first electrode comprises a conducting layer of the glove, and the second electrode comprises a conducting layer of an outer glove, wherein the disposing step includes disposing the glove substantially inside the outer glove.

15. The method of claim 13, wherein the glove includes a dielectric having a slightly conducting layer.

16. The method of claim 13, wherein the outer glove includes a dielectric having a slightly non-conducting layer.

17. The method of claim 13, wherein the second electrode comprises a non-wetting, conducting fluid. Again wetting may be ok if the fluid is safe like water

18. The method of claim 13, wherein the field generated has a strength of approximately 30 kV/cm to 50 kV/cm.

19. The method of claim 13, wherein a frequency of the high-voltage periodic signal is approximately 10 kHz to 1,000 kHz.

20. The method of claim 13, wherein an amplitude of the high-voltage periodic signal is approximately 80 V to 500 V.

21. The method of claim 13, wherein the period of time is less than approximately 1,000 ms.

22. The method of claim 13, wherein a distance between the first electrode and the second electrode is less than about 250 microns.

23. The method of claim 13, wherein the disposing step includes the steps of:
inserting the glove into a rigid enclosure, the rigid enclosure comprising the second electrode made of a deformable material and disposed within the rigid enclosure so as to define a first volume between the rigid enclosure and the second electrode; and
increasing the pressure in the first volume so as to deform the second electrode and dispose the second electrode substantially against the outer surface of the gloved hand.

24. A method of sanitizing an outer surface of a user's hand, comprising the steps of:
disposing the hand to be sanitized inside a glove having an inner and outer surface, the inner surface facing the hand; and a first electrode disposed substantially next to the outer surface of the glove;
applying a high-voltage periodic signal across the first electrode for a period of time so as to generate a field between the first electrode and the stratum corneum of the hand, the field being configured to inactivate pathogens disposed on the surface of the hand.
